(19) United States
(12) Patent Application Publication
(10) Pub. No.: US 2014/0322368 A1
(43) Pub. Date: Oct. 30, 2014

(54) METHOD OF REDUCING AND IMPROVING THE APPEARANCE OF SCAR TISSUE

(71) Applicant: NOVO SOLUTIONS MD, LLC, Paradise Valley, NV (US)

(72) Inventor: ALVIN DAVID NEEDLEMAN, Las Vegas, NV (US)

(21) Appl. No.: 14/260,945

(22) Filed: Apr. 24, 2014

Related U.S. Application Data
(60) Provisional application No. 61/816,135, filed on Apr. 25, 2013.

(51) Int. Cl. A61K 38/07; A61K 9/00; A61K 38/06; A61K 36/48; A61K 31/7048

(52) U.S. Cl. CPC A61K 38/07; A61K 36/48; A61K 31/7048; A61K 38/06; A61K 9/0014
USPC 424/757; 530/399

(57) ABSTRACT

Scars, stretch marks, keloids, keratosis, and other skin lesions can be ameliorated through topical application of a composition containing a fortified botanical complex inhibiting tryptase and chymase. A flavonoid with antioxidant properties valuable in inflammatory stress; blocking the release of histamine, inhibiting leukocytic elastase and strengthening the protective properties surrounding the extracellular matrix. Primary wound healing occurs through the release of platelet derived growth factors stimulating the production of procollagen and increasing the density of water holding skin matrix cells which provide unencumbered moisture to the skin. The formula peptides are matrikines They act in synergy to restore and maintain the skin's appearance by activating the neosynthesis of extracellular matrix molecules. As messenger molecules the matrikines are capable of regulating cell activities: they interact with specific receptors to activate certain genes involved in extracellular matrix renewal and cell proliferation. Peptides also provide a major role in restructuring and restoring collagen.

### CROSS REFERENCE TO RELATED APPLICATIONS

This patent claims priority on U.S. Provisional Patent Application No. 61/816,135, filed Apr. 25, 2013, which is incorporated herein by reference.

### FEDERALLY SPONSORED RESEARCH

Not applicable

### REFERENCES CITED U.S. PATENT DOCUMENTS

73997783 January 2006, Rosenbloom

U.S. Pat. No. 5,639,740 June 1997, Crandall

U.S. Pat. No. 5,928,659 July 1999, Moy

### FIELD OF THE INVENTION

The present invention relates to compositions and methods for the cosmetic treatment of scar tissue, stretch marks, and keloids.

### DESCRIPTION OF THE RELATED ART

Keloid and hypertrophic scarring develop as a result of a proliferation of dermal tissue following skin injury. In susceptible individuals, scars that become raised, red, and firm may result in itching and pain, and create cosmetic and functional problems. Numerous treatment modalities are available to prevent hypertrophic scars and keloids and minimize disfiguring scars from forming following a surgical procedure. No treatment regimen has been demonstrated to be universally effective.

Several modalities have been shown in past studies to have varying degrees of effectiveness in improving the healing of scars. These include pressure, occlusion, silicone, several corticosteroids, and vitamin E. A general description of the majority of the over-the-counter topical medications developed to improve the appearance of scars are liquid formulations, which combines silicone, hydrocortisone, and vitamin E, they dry rapidly and form a clear non-breathable film when applied to skin. The flexible collodion carrier adds occlusion and slight increased surface tension to deliver these multiple modalities.

Many dermatologists and plastic surgeons have incorporated products of this type into their post-surgical regimen. It has been anecdotally reported that the product may have a beneficial effect in the stimulation of endogenous collagenase production. Logically, it would seem that combining these accepted technologies would lead to improved scarring. Unfortunately, actual results are only moderately successful and the application process must be carried on for very long periods of time, more than 60 days, to produce even slight visible results.

### SUMMARY

The present invention is directed to a method of reducing and improving the appearance of scar tissue keloids and stretch marks the formulas are composed of antioxidant flavonoids, anti-proteolytic activity, peptides, botanicals, algae's, ceramides, carbohydrates and platelet derived growth factors from multiple sources autologous or homologous, including human umbilical blood, peripheral blood, fat, bone, marrow, amniotic fluid, skin, placental tissue and sources of artificial and natural cytokines and interleukins.

Scars, stretch marks, keloids, keratosis, and other skin lesions can be ameliorated or cured through topical application, to affected skin, of a cosmetic or dermatological composition containing a fortified botanical complex inhibiting tryptase and chymase. A flavonoid, with antioxidant properties that is of value in inflammatory stress; blocking the release of histamine and inhibiting leukocytic elastase and strengthening the protective properties surrounding the extracellular matrix. Primary wound healing is accomplished through the release of platelet derived growth factors stimulating the production of procollagen and increasing the density of water holding skin matrix cells which provide unencumbered moisture to the skin. The formula peptides are matrikines (extracellular matrix-derived peptides which regulate cell activity) they act in synergy to restore and maintain the skin's appearance by activating the neosynthesis of extracellular matrix molecules. As messenger molecules the matrikines are capable of regulating cell activities: they interact with specific receptors to activate certain genes involved in extracellular matrix renewal and cell proliferation. Peptides also provide a major role in restructuring and restoring collagen.

New modalities for treatment of both hypertrophic, atrophic scars and keloids have improved the cosmetic surgeon's approach to the management of this commonly faced problem, which is often of great concern to patients in the postoperative setting. Conventional as well novel approaches including lasers, silicone pressure dressings, immune modulator therapy and recently developed combination products have appeared. However, the invention presents a well-founded technology expanded to include a composite of the empirical data from new research approaches to the physiological causes of scar keloid and stretch mark formation. Research- development and intense field trials and studies resulted in a novel approach to ameliorating and curing scars, stretch marks and keloids. The invention demonstrates the success of combining growth factors, carbohydrate derived moisturizers, anti-proteolytic activity, an antioxidant flavonoid with the collagen stimulating properties of peptides, botanicals, and a mast cell inhibitor leading to visible and measurable reduction of both young and older scars, stretch marks and keloids. An additional focus of the invention is the relief of “wound tension.”

Wound tension; pressure exerted between opposing wound surfaces is a major cause of scar formation and the stimulation of keloid tissue in “keloid-prone” individuals. One of the major actions of the invention is directed at tension relief by introducing an inter-tissue osmotic moisture flow. A complex carbohydrate induces immediate (on contact) tissue moisturizing, softening and increasing elasticity . . . extremely more effective than the use of occlusive films i.e., silicones, modified colloidians.

### DETAILED DESCRIPTION OF THE INVENTION

Formula rational for the invention: the following active ingredients were combined for their complementary actions, a green bean extract, an inhibitor of trypsin and chymotrypsin, an antioxidant, rutin, which contributes to stabilizing mast cells and to the anti-elastase properties, two peptides already having demonstrated their efficacy in the reconstitution of the extracellular matrix and a resurgence of collagen levels. The dry green bean extract or Phaseolus lunatus contains a small protein with a molecular weight of 8,000 Daltons which inhibits tryptase and chymase. This small, cysteine-rich protein is stabilized by disulfide bridges which are important in terms of stability and efficacy. Its three-dimensional structure has 2 trypsin and chymotrypsin recognition sites, thus conferring an inhibitory effect on the protein. The protein is a member of the Bowman-Birk protease inhibitor class, the leading protein of which is the soybean Bowman-Birk protease inhibitor (WARE, 1997). Three-dimensional structure of the BBI protein extracted from Phaseolus. In the skin, collagenase activity depends on the conversion of procollagenase into active collagenase. Conversion is dependent on trypsin. Inhibiting trypsin thus limits collagenase activity. It is also known that MMP2 and MMP9 are activated by trypsin (IBA, 2004). Chymotrypsin, normally present in the skin (EGELRUD, 1993) and abundantly released by mast cells, is to be markedly reduced during cell cicatrization (ALGERMISSEN et al., 1999) in order to promote de novo synthesis of the matrix.

Under those conditions, a dual trypsin-chymotrypsin inhibitor enables restoration of tissue homeostasis.

Rutin was based on the latter's antioxidant properties of value in inflammatory stress and, above all, its ability to stabilize mast cells as shown by recent studies: in the presence of rutin, activated mast cells no longer release histamine (CHEN, 2000). The surface antigen presentation which reflects mast cell activation is antagonized by rutin (GONG et al., 2003). Moreover, identified a new property of rutin: inhibition of leukocytic elastase. This strengthens the protective properties with respect to the extracellular matrix. Lastly, 2 peptides (bio peptides palmitoyl-GQPR and palmitoyl-GHK), which activate the genes involved in de novo matrix synthesis (particularly collagen and fibronectin) and which promote proliferation of keratinocytes and fibroblasts and proteoglycan anchoring, were selected to promote repair of damaged matrix.

In some respects the invention may appear to contradict certain established practices, i.e., “Decreased blood flow with consequent decrease in alpha 2-macroglobulin and subsequent increase in collagenase-mediated collagen breakdown, normally inhibited by alpha 2-macroglobulin. Lower levels of chondroitin sulfate yielding increased collagen degradation. Decreased scar hydration yielding mast cell stabilization and subsequent diminished neovascularization and extra-cellular matrix production. Hypoxia leading to fibroblast degeneration and collagen degradation”. However, the geometrically organized reconstruction of collagen and the release of wound specific growth factors generates increased quantities of new procollagen and newly well-formed architecture within the skin matrix provides a more physiologically sound repair, faster onset of activity and a shorter time interval for improvement both physically and visually.

Formula rational: Butylene glycol-Water (Aqua)-Cetyl hydroxyethylcellulose-Rutin-Palmitoyl Oligopeptide-Palmitoyl Tetrapeptide-7-Phaseolus Lunatus (Green Bean) Extract Demonstrated activity in vitro Inhibition of proteolytic enzymes (2% of the ingredient)
−7% to −15% inhibition of chymotrypsin
−14% to −58% inhibition of trypsin
−66% to −90% inhibition of elastase
A protective effect enabling 63% fibroblast adhesion to the extracellular matrix even in the presence of trypsin.

Synthesis of matrix proteins by fibroblasts in the presence of 2% of the ingredient.
+102% for collagen I and +27% for collagen IV
+91% for fibronectin

In vivo Clinical trial on 13 subjects with scarring Approximately 4 months old. The subjects applied 2% of the ingredient for 2 months:
an 11% increase in dermal thickness
a 72% decrease in scar depth associated with restoration of a normal dermis as imaged by ultrasound
dermatologist's assessment of a significant improvement in the color (−22%), rough appearance (−22%) and scar width (−27%), considered. The differences were very significant

Toxicology (as per the UNITIS Charter): Patch-test on humans (10 subjects)
HET CAM
RIPT on 50 subjects
Ames' test
Expert certification, no irritation observed

REVIEW: fibroblasts are physically disconnected from their matrix and their role in the denovo synthesis of dermal macromolecules is antagonized by the presence of proteolytic enzymes. The enzymes derive locally from infiltration of specialized leukocytes, particularly mast cells and, subsequently, macrophages. The fibroblasts are overwhelmed. (1) Limiting, to the greatest extent possible, the effect of mast cell degranulation releasing trypsin and chymotrypsin and offsetting leukocytic elastolysis elastase released by macrophages) is thus considered crucial. (2) Helping fibroblasts to repair the matrix also remains indispensable. The concept is for decreasing Scar emergence and is based on the above analysis.

Thus, the following active ingredients were combined for their complementary actions:
a green bean extract, an inhibitor of trypsin and chymotrypsin,
an antioxidant, rutin, which contributes to stabilizing mast cells and to the antielastase properties, —two peptides already having demonstrated their efficacy in the reconstitution of the extracellular matrix.

The dry green bean extract or Phaseolus lunatus contains a small protein with a molecular weight of 8,000 Daltons which inhibits tryptase and chymase. This small, cysteine-rich protein is stabilized by disulfide bridges which are important in terms of stability and efficacy.

Its three-dimensional structure has 2 trypsin and chymotrypsin recognition sites, thus conferring an inhibitory effect on the protein. The protein is a member of the Bowman-Birk protease inhibitor class, the leading protein of which is the soybean Bowman-Birk protease inhibitor (WARE, 1997).

### Three-Dimensional

The decision to add rutin was based on the latter's antioxidant properties of value in inflammatory stress and, above all, its ability to stabilize mast cells as shown by recent studies: in the presence of rutin, activated mast cells no longer release histamine (CHEN, 2000). The surface antigen presentation which reflects mast cell activation is antagonized by rutin (GONG et al., 2003).

Moreover, the importance of a recognition of a new property of rutin: inhibition of leukocytic elastase. This strengthens the protective properties with respect to the extracellular matrix. Lastly, a silicone cross polymer allows selective respiration passing oxygen and moisture over the affected area and 2 peptides (biopeptides palmitoyl-GQPR and palmitoyl-GHK), which activate the genes involved in de novo matrix synthesis (particularly collagen and fibronectin) and which promote proliferation of keratinocytes and fibroblasts and proteoglycan anchoring, were selected to promote repair of damaged matrix.

One aspect of the present invention is directed to a relief of wound tension through an inter-tissue osmotic moisture flow.

Another aspect of the present invention is directed to an amelioration or cure of scar tissue.

Another aspect of the present invention is directed to an amelioration or cure of older scar tissues.

Another aspect of the present invention is directed to an amelioration or cure of keloids.

Another aspect of the present invention is directed to an amelioration or cure of older keloids.

Another aspect of the present invention is directed to an amelioration or cure of stretch marks.

Another aspect of the present invention is directed to an amelioration or cure of old stretch marks.

Another aspect of the present invention is directed to the restoration of fibroid infiltrated tissue to physiologically active and architecturally compatible tissue.

Another aspect of the present invention is directed to the wound healing activity of growth factors.

Another aspect of the present invention is directed to the activity of formula matrikines (Peptides) to regulate cellular activity by interacting with specific receptors to activate certain genes involved in extra cellular matrix renewal and cell proliferation.

Another aspect of the present invention is directed to the synergistic action of multiple growth factors with other proteins in the epidermis and the dermis stimulating skin repair and regeneration.

Another aspect of the present invention is directed to a formula(s) containing a balanced mixture of growth factors, peptides, anti proteolytic activity, antioxidant flavonoid, cross polymers, botanicals, ceramides and absorption systems that repair, restore and mollify the appearance and the skin architecture of scars, stretch marks and keloids.

Another aspect of the present invention is directed to the formation of a cross polymer breathable barrier for the balanced flow of oxygen and moisture over the affected scar-stretch marks-keloid area.

Another aspect of the present invention is directed to the topical application of a prepared cream-lotion-spray or serum containing the invention formula(s)

1. What is claimed in the invention is the activity of formula matrikines (Peptides) to regulate cellular activity by interacting with specific receptors to activate certain genes involved in extra cellular matrix renewal and cell proliferation.

2. What is claimed in the invention is the synergistic action of multiple growth factors with other proteins in the epidermis and the dermis stimulating skin repair and regeneration

3. What is claimed by the invention is a formula(s) containing a balanced mixture of growth factors, peptides, anti proteolytic activity, antioxidant flavonoid, cross polymers, botanicals, ceramides and absorption systems that repair, restore and mollify the appearance and the skin architecture of scars, stretch marks and keloids.
